(19) United States
(12) Patent Application Publication
(10) Pub. No.: US 2021/0023520 A1
(43) Pub. Date: Jan. 28, 2021

(54) PRODUCTION OF NANOPARTICLES

(71) Applicant: Analytik Jena AG, Jena (DE)

(72) Inventor: Christian Paulenz, Jena (DE)

(21) Appl. No.: 16/931,939

(22) Filed: Jul. 17, 2020

(30) Foreign Application Priority Data
Jul. 24, 2019 (DE) 10 2019 120 020.2

(51) Int. Cl. B01J 2/06; A61K 9/51

(52) U.S. Cl. CPC B01J 2/06; B82Y 5/00; A61K 9/5192

(57) ABSTRACT

The present disclosure relates to an apparatus and to a method for production, especially continuous production, of nanoparticles, including a line to convey water with a predeterminable flow velocity and a system arranged orthogonally to the line for introducing at least one dissolved substance into the line for producing the nanoparticles.

### CROSS-REFERENCE TO RELATED APPLICATION

The present application is related to and claims the priority benefit of German Patent Application No. 10 2019 120 020.2, filed on Jul. 24, 2019, the entire contents of which are incorporated herein by reference.

### TECHNICAL FIELD

The present disclosure relates to an apparatus for production, especially continuous production, of nanoparticles, as well as to a corresponding method.

### BACKGROUND

Nanoparticles are assemblages of atoms or molecules with nanometer size, especially in the range between 1-100 nm. Nanoparticles are defined in the standard, ISO/TS 27687:2008, as three-dimensional nano-objects.

Known in the state of the art are the most varied of types of nanoparticles, for example, nanoparticles containing carbon, metals, semimetals, semiconductors, zeolites or polymers. The physical and chemical properties of nanoparticles differ, as a rule, significantly from macroscopic solid bodies of the same materials. Nanoparticles are applied in a wide variety of technological fields. They are used, for example, in nano electronics, as nano materials for producing the most varied of products, for medicines and even in environmental technology. In the case of medicines, they serve, among other things, for targeted dispensing of medicines and for diagnostics, such as described, for example, in EP0740548B1 or WO2013/160773A1.

Depending on the contemplated application of nanoparticles, an exactly defined size distribution is required. The particular size distribution depends, in such case, frequently on the method used for producing the nanoparticles. Accordingly, the state of the art displays the most varied of production process.

On the one hand, numerous mechanical-physical production processes are known, such as the so-called top-down processes, for example, various grinding processes or lithographic methods. Often, however, also chemical-physical approaches are used, which make use of molecular, or atomic self-organization and fall under the label, bottom-up processes. Examples of the latter are the aerosol or gas phase methods, precipitation reactions, or precipitation processes, and sol-gel processes. The article “Role of Surfactants in Nanotechnology and their Applications” by M. I. Salwan et al., published in Int. J. Curr. Microbiol. App. Sci. (2014) 3(5): 237-260 provides, additionally, an overview of different techniques for producing nanoparticles in an emulsion method. In “A simple microfluidic apparatus for fabrication of double emulsion droplets and polymer microcapsules” by G. Nurumbetov et al., published in Polym. Chem., 2012, 3, 1043, additionally, a method for producing double emulsions is described.

For industrial use of nanoparticles, methods must be available for producing significant amounts of nanoparticles of constant quality. Also, the costs of the methods and utilized materials can play a role.

Known from U.S. Pat. No. 8,211,205B1, for example, is a scale-up method for producing nanoparticles by applying inorganic metal salts. Also, US2009/0074655A1 describes techniques for producing nanoparticles by means of a sol-gel method with application of inorganic metal salts.

U.S. Pat. No. 9,956,179B2 describes a method as well as an apparatus for production of nanoparticles from amphiphilic copolymers with controllable size and yield.

Known from “Fabrication of PLGA nanoparticles with a fluidic nanoprecipitation system” by H. Xie et al., Journal of Nanobiotechnology, 2010, 8:18 is a precipitation method for production of nanoparticles from PLGA, with which a uniform size distribution of the particles can be achieved, as well as size of the produced particles set according to requirements.

### SUMMARY

Starting from the state of the art, an object of the present disclosure is to provide an apparatus, and a method, for producing the most varied of nanoparticles in large amounts and with constant quality.

The object is achieved by the apparatus as defined in claim 1 as well as by the method as defined in claim 12.

As regards the apparatus, the object is achieved by an apparatus for production, especially continuous production, of nanoparticles, comprising a line for conveying water with a predeterminable flow velocity, and a system arranged at a predeterminable angle, especially orthogonally, to the line for introducing at least one dissolved substance into the line for producing the nanoparticles.

The substance applied for producing the nanoparticles is, thus, introduced into a water conveying line. The line is, for example, a tube, or a pipeline or a hose. Preferably, the line extends at least sectionally horizontally. The flow velocity is preferably at least at times constant. The apparatus can further include a collection vessel for catching the formed nanoparticles and/or a reservoir for the dissolved substance.

With the solution of the present disclosure, a continuous production of nanoparticles can be enabled, which avoids the disadvantages of batch-based production processes. Because of the direct introduction of the substance serving for producing the nanoparticles into a continuous, and an as pulsation free as possible, transverse flow of the water in the line, a very homogeneous, liquid spray effect is produced, which finely distributes the substance. In such case, the solvent decrease for each introduced droplet is basically identical.

With the apparatus of the present disclosure, the most varied of nanoparticles can be produced. The solvent applied for dissolving the substance is preferably matched to the sub stance.

An embodiment includes that a surface active substance is addable, or added, to the water.

Another embodiment includes that the system for introducing the at least one dissolved substance comprises a pipette tip or a supply line, especially a tube or a capillary. In such case, the line for conveying water with a predeterminable flow velocity and the system for introducing fluid are advantageously connected together. Especially, the line can have an opening in the wall, or two portions of the line can be joined to form the line by means of a connecting piece, for example, a T piece, wherein the fluid connection is created via the connecting piece.

Another especially preferred embodiment includes that at least two systems are present, each for introducing at least one dissolved substance, wherein a first system serves for introducing a first dissolved substance and a second system for introducing a second dissolved substance. The two systems can, on the one hand, be arranged separately from one another at different positions relative to the line. It is, however, likewise an option to combine the two systems.

Especially, an embodiment of the apparatus with two systems includes the feature that the second system at least partially coaxially surrounds the first system. In this way, a homogeneous mixing of the two substances can be achieved, before the substances are introduced into the water conveying line.

Such an embodiment of two systems for introducing two substances into a line, in the case of which the one system coaxially surrounds the other, is suited also for an apparatus for production of nanoparticles, in the case of which instead of a water conveying line a glass beaker is used, into which the substances are introduced.

It is, furthermore, advantageous that the first and second systems be fluidically connected together. The two systems are especially embodied and/or arranged in such a manner that an opening of the first system communicates with an internal volume of the second system, and wherein the two substances are introduced together into the line through an opening of the second system. Preferably, the at least two substances are, thus, introduced into the line simultaneously and together.

In an embodiment, at least one substance is a polymer, a medicine, a DNA, an RNA, a protein or mixture, especially of one of these substances and a transport means.

Another embodiment includes that the apparatus comprises at least one pump, which serves to set the predeterminable flow velocity and/or to set a predeterminable introduction speed for the at least one substance into the line. Preferably, the apparatus includes at least one pump, with which the predeterminable flow velocity can be set and, in each case, one pump per applied system for introducing a substance for producing the nanoparticles.

In an especially preferred embodiment, the line is embodied in such a manner that in a region, in which the system for introducing the at least one substance into the line is arranged, a flow profile reigning in the line is changed compared with sections of the line lying outside this region, especially wherein a turbulent flow profile is present in the region. The turbulent flow profile provides a tearing of the substance from the system for introducing the substance. Since the flow profile in the region of the system is turbulent, an undesired droplet formation of the substance can be prevented in this region.

Advantageously in this regard, the system for introducing the at least one substance extends to a predeterminable penetration depth, especially protrudes inwardly a predeterminable penetration depth of up to 50% of a diameter of the line, into the line. By such an arrangement, a turbulent flow profile can be achieved in especially easy manner in the region of the system.

Finally, the apparatus includes, in yet another embodiment, means to set a predeterminable temperature of the apparatus at least in a region, in which the system for introducing the at least one substance into the line is arranged. By setting a predeterminable temperature, a desired distribution of the particle size of the nanoparticles can be set.

The object underlying the present disclosure is achieved, furthermore, by a method for production, especially continuous production, of nanoparticles, comprising method steps as follows: conveying water in a line with a predeterminable flow velocity; and

introducing at least one dissolved substance into the line by means of a system arranged at a predeterminable angle, especially orthogonally, to the line, for introducing the at least one dissolved substance into the line for producing the nanoparticles.

Because of the direct introduction of the substance serving for producing the nanoparticles into a continuous and an as pulsation free as possible, transverse flow of the water in the line, advantageously a very uniform liquid spray effect is produced, which finely distributes the substance. In such case, the solvent decrease is basically identical for each introduced droplet.

In an embodiment of the method, the nanoparticles are produced in a precipitation method or in an emulsion method.

If the nanoparticles are produced, for example, by means of a precipitation process, then there is in the line a decrease of the solvent concentration and, associated therewith, a precipitation of the substance.

In the case of an emulsion method, in contrast, the solvent concentration increases in the reaction space with progressive introduction.

In an especially preferred embodiment of the method, flow velocity is set as a function of desired size of the nanoparticles. Advantageously, thus, nanoparticles of different type and different size can be produced with the present disclosure.

Another embodiment of the method includes that at least two substances are introduced with the aid of two systems for introducing dissolved substances into the line, wherein the second system at least partially coaxially surrounds the first system, and wherein a second introduction speed for the second substance is greater than or equal to a first introduction speed for the first substance because of the setting of the flow velocities of the two substances

It is to be noted here that the embodiments described in connection with the apparatus of the present disclosure can be used mutatis mutandis also for the method of the present disclosure, and vice versa.

### BRIEF DESCRIPTION OF THE DRAWINGS

The present disclosure will now be explained in greater detail based on the appended drawing, the figures of which show as follows:

FIGS. 1a and 1b show embodiments of an apparatus of the present disclosure having a line and a system for introducing the at least one substance;

FIG. 2 shows a further embodiment of an apparatus of the present disclosure having a line and a system for introducing the at least one substance;

FIGS. 3a-3c show embodiments of an apparatus of the present disclosure with two systems for introducing two substances;

FIG. 4 shows an embodiment for providing a turbulent flow profile in the region of the system; and

FIG. 5 shows an embodiment of the apparatus of the present disclosure with means to set a predeterminable temperature in the region of the system.

In the figures, equal elements are provided with equal reference characters.

### DETAILED DESCRIPTION

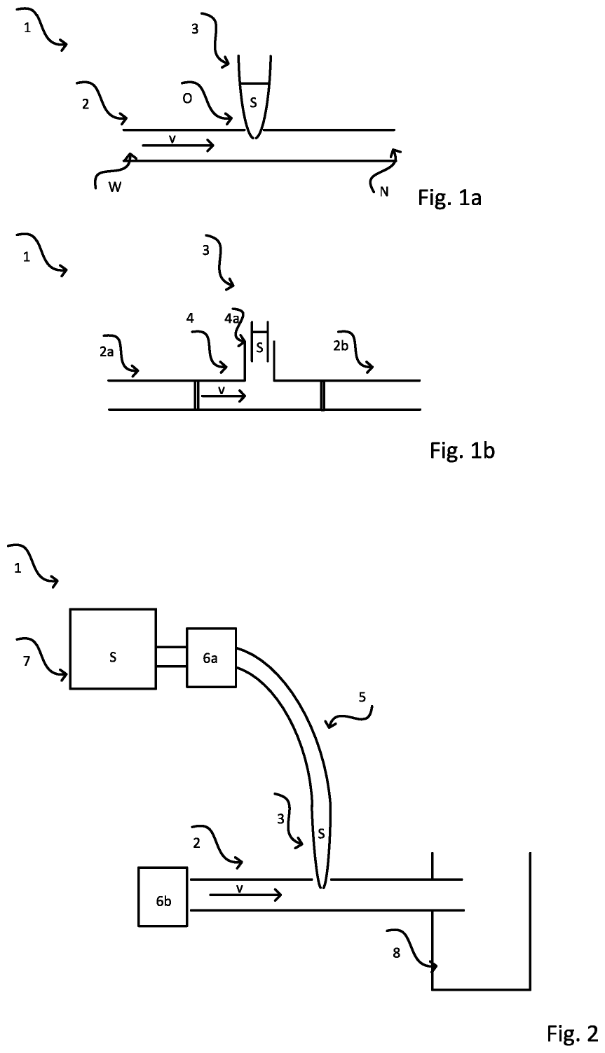

FIG. 1 shows two possible embodiments of an apparatus 1 of the invention having, in each case, a line 2 flowed through by water W and a system 3 arranged at a predeterminable angle, here, by way of example, orthogonally, to the line for introducing into the line 2 a dissolved substance S, which serves for production of the nanoparticles N. The substance S is, for example, a polymer, a medicine, a DNA, an RNA, a protein or a mixture, especially of one of these substances S and a transport means. The substance S is dissolved in a suitable solvent. The system 3 according to the embodiment of FIG. 1a includes a pipette tip, and according to the embodiment of FIG. 1b, in contrast, a capillary. The dissolved substance S is located in the system 3. Besides the two examples of embodiments for a system 3 in the sense of present invention, numerous other embodiments are possible, which likewise fall within the scope of the present invention.

Line 2 is flowed through by water W with a predeterminable flow velocity v. Line 2 is, for example, a hose or a pipeline. In the embodiment of FIG. 1a, the line has in the region of a wall an opening O, through which system 3 is introduced into the line 2. In the case of FIG. 1b, the line is composed of two line portions 2a, 2b, which are connected via the connecting piece 4. The system 3 is introduced into the line 2 via the connecting piece 4; in particular, via the opening O′. The connecting piece is a T piece in the illustrated embodiment. It is, however, also possible in the context of the present invention to use other connecting pieces 4, for example, connection crosses, or elbows.

The nanoparticles N are produced by introduction of the substance S into the line 2. Preferably, a precipitation method or an emulsion method is used. Advantageously because of the continuous introduction of the dissolved substance S into the water conveying line 2, a uniform liquid spray effect occurs, which finely distributes the mixture, and, in the case of a precipitation method, for example, upon the decrease of the solvent concentration in the transverse flow, leads to precipitation. This solvent decrease is, in contrast to the case, in which a glass beaker is used, essentially identical for each introduced drop of the dissolved substance S. Advantageously, the nanoparticles N can be continuously produced with the present invention. Advantageously furthermore, application of an apparatus 1 of the invention enables direct connection with a clean-up unit (not shown) arranged in an end region of the line 2. The production of nanoparticles N can occur both with as well as also without application of surface active substances. Moreover, the substance S can be present both in a homogeneous as well as also in an inhomogeneous solution.

FIG. 2 shows another possible embodiment of an apparatus 1 of the invention. In supplementation of the variants shown in FIG. 1, the apparatus 1 of FIG. 2 has a reservoir 7, which contains a supply of the dissolved substance S, and a pump 6a and a supply line 5 connected to the system 3 for introducing the substance S into the line 2. Arranged in the line 2 is another pump 6b, which serves to set the predeterminable flow velocity v. Arranged at the end of the line 2, furthermore, is a collection vessel 8 for receiving the formed nanoparticles. These additional components are, however, not absolutely necessary. Alternatively only one or a selection of the components supplementally shown in FIG. 2 can be used. Also, an option is, for example, that at least two pumps 6 are arranged in the region of the line 2 and/or in the region of the system 3.

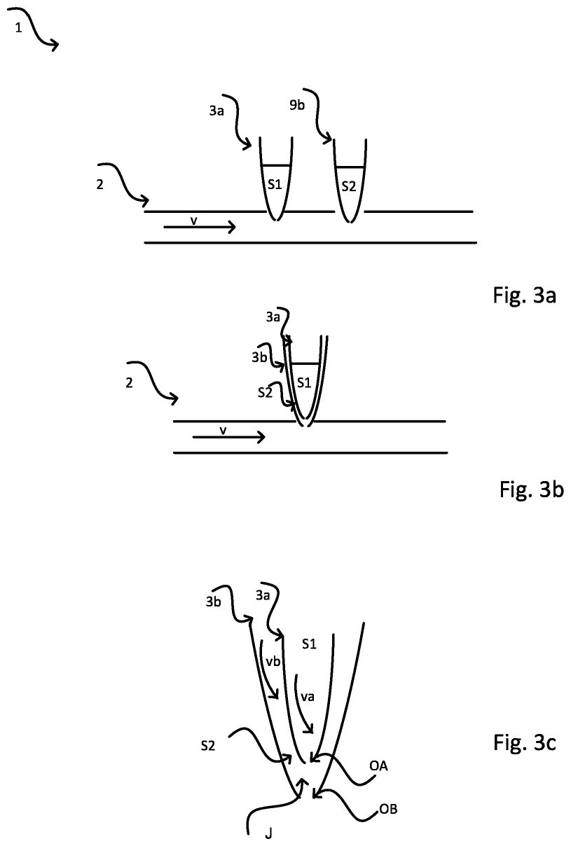

In other embodiments, the apparatus 1 of the invention includes two systems 3a, 3b for introducing at least two substances S1 and S2 into the line 2. Two examples of such variants of the present invention are shown in FIG. 3. Thus, shown in FIG. 3a are two equally constructed systems 3a, 3b, each of which corresponds to the embodiment shown in FIG. 1a. In such case, the two substances S1 and S2 are introduced into the line 2 offset from one another.

In the case of the embodiment of FIG. 3b, in contrast, the second system 3b surrounds the first system 3a coaxially. In this way, the two substances S1 and S2 are first mixed with one another, before they reach the line 2. In the case of a precipitation process, it is possible, in this manner, for example, to prevent a premature precipitation of the substances S1, S2. This construction is shown in greater detail in FIG. 3c.

The second system 3b coaxially surrounds the first system 3a and is fluidically connected with the first system 3a. The opening OA of the first system 3a communicates with an internal volume of the second system 3b. Thus, the first substance S1 is introduced into the second substance S2 at the junction J. The two substances S1, S2 are then introduced into the line through the second opening OB. Advantageously, the flow of the two substances S1, S2 at the junction J is straightened and stabilized supplementally by a concentric arrangement of the first system 3a relative to the second system 3b. In this way, a very uniform introduction of the substances S1, S2 into the line 2 can be assured. Other important parameters, which concern the type of introduction of the substances S1, S2 into the line 2, are the introduction speeds va and vb of the two substances S1, S2 into the two systems 3a, 3b. Thus, advantageously the second introduction speed vb for the second substance S2 is greater than or equal to the first introduction speed va for the first substance S1.

FIG. 4 shows an embodiment of an apparatus 1 of the invention, which assures a turbulent flow profile in the region B surrounding the system 3. A turbulent flow profile in this region B leads to a tearing off of the at least one substance S and avoids an undesired droplet formation in the region of the line 2. Such a sectionally turbulent flow profile can be achieved in many different ways, for example, by a sectional change of the cross-sectional area of the line or by the introduction of a flow resistance, for example, in the form of a flow body, into the line. An especially easy way of producing a turbulent flow profile in the region B is shown in FIG. 4. The embodiment shown there corresponds largely to the embodiment shown in FIG. 1b. However, the system 3 protrudes to a penetration depth d into the line 2. This serves as a flow resistance for the system 3, so that a turbulent flow profile is present in the region B.

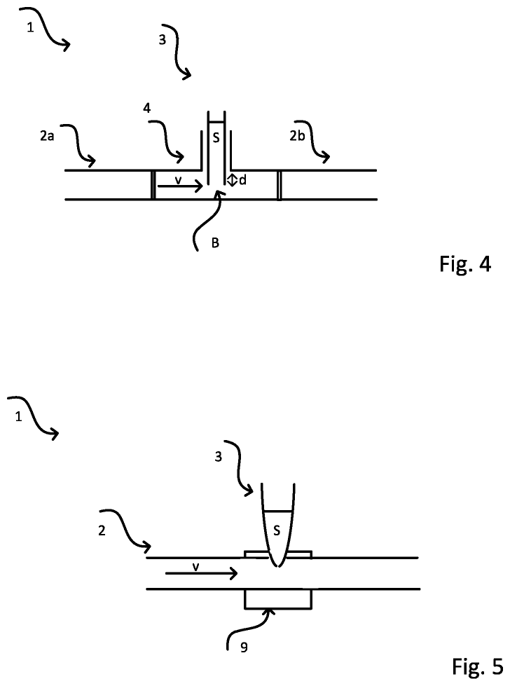

Another embodiment of the present invention is shown in FIG. 5. This embodiment corresponds essentially to the variant illustrated in FIG. 1a. Additionally, the apparatus 1 of FIG. 5 includes, however, means 9 to set a predeterminable temperature T of the apparatus 1 in the region B. The means 9 to set the predeterminable temperature T comprises, for example, at least one heating unit for heating the region B and a temperature sensor for sensing the temperature T reigning in the region B.

1. An apparatus for continuous production of nanoparticles, the apparatus comprising:
a line adapted to convey water at a flow velocity; and
an introduction system arranged at an angle to the line and configured to introduce at least one dissolved substance into the line such that nanoparticles are producing in the flowing water.

2. The apparatus of claim 1, wherein the at least one dissolved substance is a surface active sub stance.

3. The apparatus of claim 1, wherein the introduction system comprises a pipette tip, a tube or a capillary.

4. The apparatus of claim 1, wherein the introduction system arranged at an orthogonal angle to the line.

5. The apparatus of claim 1, wherein the introduction system comprises at least two systems, each configured to introduce at least one dissolved substance, wherein a first system is configured to introduce a first dissolved substance and a second system is configured to introduce a second dissolved substance.

6. The apparatus of claim 5, wherein the second system at least partially coaxially surrounds the first system.

7. The apparatus of claim 5, wherein the first system and the second system are fluidically connected together.

8. The apparatus of claim 1, wherein at least one substance of the at least one dissolved substance is a polymer, a medicine, a DNA, an RNA, a protein or a mixture, wherein the mixtures includes the at least one substance and a transport medium.

9. The apparatus of claim 1, further comprising at least one pump adapted to effect the flow velocity of the water and/or to introduce the at least one substance into the line via the introduction system at an introduction speed.

10. The apparatus of claim 1, wherein the line includes a region in which the introduction system is arranged, wherein the line is configured in the region such that a flow profile in the region of the line is different relative to sections of the line outside the region.

11. The apparatus of claim 10, wherein the flow profile in the region is a turbulent flow profile.

12. The apparatus of claim 10, wherein the introduction system extends into the line to a penetration depth that is up to 50% of a diameter of the line.

13. The apparatus of claim 1, further comprising a heating unit configured to set a temperature of the apparatus at least in a region in which the introduction system is arranged.

14. A method for continuous production of nanoparticles, the method comprising:
conveying water in a line at a flow velocity; and
introducing at least one dissolved substance into the line via an introduction system arranged at an angle to the line, the introduction system configured to introduce at least one dissolved substance into the line such that nanoparticles are producing in the flowing water.

15. The method of claim 14, wherein the at least one dissolved substance is introduced into the line such that the nanoparticles are produced by a precipitation method or by an emulsion method.

16. The method of claim 14, wherein flow velocity is a function of a desired size of the nanoparticles.

17. The method of claim 14, wherein a first substance and a second substance are introduced into the line via the introduction system, which comprises a first system and a second system, each configured to introduce dissolved substances into the line, wherein the second system at least partially coaxially surrounds the first system, and wherein a second introduction speed of the second substance is greater than or equal to a first introduction speed of the first substance.

18. The method of claim 14, further comprising generating a turbulent flow profile in the line in a region of the line in which the introduction system is arranged.

19. The method of claim 14, further comprising heating a region of the line in which the introduction system is arranged to a desired temperature.

20. The method of claim 14, wherein at least one substance of the at least one dissolved substance is a polymer, a medicine, a DNA, an RNA, a protein or a mixture, wherein the mixtures includes the at least one substance and a transport medium.
